(19) United States
(12) Patent Application Publication
(10) Pub. No.: US 2021/0162237 A1
(43) Pub. Date: Jun. 3, 2021

(54) RADIOTHERAPY APPARATUS AND CONTROL METHOD THEREOF

(71) Applicants: OUR UNITED CORPORATION, Shaanxi (CN); SHENZHEN OUR NEW MEDICAL TECHNOLOGIES DEVELOPMENT CO., LTD., Shenzhen (CN)

(72) Inventors: Peng Zan, Shaanxi (CN); Hao Yan, Shaanxi (CN)

(21) Appl. No.: 17/152,421

(22) Filed: Jan. 19, 2021

Related U.S. Application Data
(63) Continuation of application No. PCT/CN2018/105843, filed on Sep. 14, 2018.

(51) Int. Cl. A61N 5/10; A61B 6/00; G01T 1/16

(52) U.S. Cl. CPC A61N 5/1067; A61N 5/1081; A61B 6/482; A61N 2005/1089; A61B 6/4441; A61B 6/4208; G01T 1/16; A61B 6/54

(57) ABSTRACT

A radiotherapy apparatus includes a rotating gantry rotatable about a central axis and a multi-energy imaging device. The multi-energy imaging device includes an imaging source and an imager. The imaging source is configured to generate X-rays of at least two energy levels and emit X-rays of at least one energy level in the X-rays of at least two energy levels, so that the X-rays of at least one energy level pass through a site to be treated of a patient. The X-ray of at least one energy level is configured to meet imaging requirements of the site to be treated. The imager is configured to receive the X-rays of at least one energy level that pass through the site to be treated, and to generate X-ray images of at least one energy level of the site to be treated according to the X-rays of at least one energy level. The imaging source and the imager are arranged opposite to each other on the rotating gantry.

### CROSS-REFERENCE TO RELATED APPLICATIONS

This application is a bypass continuation application of International Patent Application No. PCT/CN2018/105843 filed on Sep. 14, 2018, which is incorporated herein by reference in its entirety.

### TECHNICAL FIELD

The present disclosure relates to the field of radiation therapy, and in particular, to a radiotherapy apparatus and a control method thereof.

### BACKGROUND

In modern society, image guided radiation therapy (IGRT) technology is used for capturing images during fractionated treatment setup and/or a treatment, so as to use these images to guide the treatment and/or subsequent fractionated treatments. During each fractional treatment, an image of a patient after setup is obtained by an imaging device, and then the image is registered with a reference image (e.g., a digitally reconstructed radiography (DRR) image obtained through a computed tomography (CT) scan) in a treatment plan system to obtain a setup error. Then, a position of a target area of the patient is adjusted according to the setup error, so as to achieve precise treatment of the target area of the patient.

Radiotherapy devices in current IGRT systems can only generate X-rays of a single energy level for imaging. X-rays of high energy levels (100 KV-6 MV) are very penetrating, and therefore can be used to form clear images of high-density targets such as bones. However, an imaging effect of X-rays of high energy levels is poor for low-density targets such as soft tissues. Conversely, X-rays of low energy levels (50-100 KV) are less penetrating, and can be used to form clear images of low-density targets such as soft tissues. Therefore, existing radiotherapy devices cannot meet different needs of different body tissues of the patient for X-ray imaging during radiation therapy, which may lead to inaccurate positioning of a site to be treated of the patient during radiation therapy and thus affect an accuracy and effect of radiotherapy.

### SUMMARY

Embodiments of the present disclosure provide a radiotherapy apparatus and a control method thereof, which are used to meet different needs of different body tissues of a patient for X-ray imaging during radiotherapy, and can position a site to be treated of the patient more accurately and improve accuracy and effect of radiotherapy.

In order to achieve the above object, the following technical solutions in embodiments of the present disclosure are adopted.

In a first aspect, a radiotherapy apparatus is provided. The radiotherapy apparatus includes a rotating gantry rotatable about a central axis and a multi-energy imaging device. The multi-energy imaging device includes an imaging source and an imager. The imaging source and the imager are arranged opposite to each other on the rotating gantry. The imaging source is configured to generate X-rays of at least two energy levels and emit X-rays of at least one energy level in the X-rays of at least two energy levels, so that the X-rays of at least one energy level pass through the site to be treated of the patient. The imager is configured to receive the X-rays of at least one energy level that pass through the site to be treated, and to generate X-ray images of at least one energy level of the site to be treated according to the X-rays of at least one energy level.

In a second aspect, another radiotherapy apparatus is provided. The radiotherapy apparatus includes a rotating gantry rotatable about a central axis, and a multi-energy imaging device and at least one processor disposed on the rotating gantry. The multi-energy imaging device includes an imaging source and an imager. The at least one processor is configured to control the imaging source to obtain at least one target X-ray image in conjunction with the imager. The at least one target X-ray image is at least one of X-ray images of at least one energy level of a site to be treated. The at least one target X-ray image is registered with at least one pre-stored reference image. A positional deviation of the site to be treated is obtained according to a result of registering the at least one target X-ray image with the at least one reference image.

In a third aspect, a control method of the radiotherapy apparatus is provided. The control method includes: controlling an imaging source to obtain at least one target X-ray image in conjunction with an imager; registering the at least one target X-ray image with at least one pre-stored reference image; and obtaining a positional deviation of a site to be treated of a patient according to a result of registering the at least one target X-ray image with the at least one reference image.

In the radiotherapy apparatus and the control method thereof provided in embodiments of the present disclosure, the radiotherapy apparatus includes the rotating gantry rotatable about the central axis and the multi-energy imaging device. The multi-energy imaging device includes an imaging source and an imager. The imaging source and the imager are arranged opposite to each other on the rotating gantry. The imaging source is configured to generate X-rays of at least two energy levels and emit X-rays of at least one energy level in the X-rays of at least two energy levels, so that the X-rays of at least one energy level pass through the site to be treated of the patient. The imager is configured to receive the X-rays of at least one energy level that pass through the site to be treated and generate X-ray images of at least one energy level of the site to be treated according to the X-rays of at least one energy level. Therefore, in a case where there is a need to position the site to be treated of the patient, it may be possible to control the imaging source to generate at least one target X-ray image in conjunction with the imager. Then, the at least one target X-ray image may be registered with the at least one pre-stored reference image. Finally, the positional deviation of the site to be treated may be obtained according to the result of registering the at least one target X-ray image with the at least one reference image. After the positional deviation of the site to be treated is obtained, a current position of the site to be treated relative to a treatment bed may be determined according to the positional deviation. Therefore, it may be possible to determine whether the treatment bed should be adjusted to change a position of the patient, so the radiotherapy goes smoothly. In the technical solutions provided in the embodiments of the present disclosure, the imaging source can generate X-rays of various energy levels. In this way, X-ray images of different energy levels can be generated to meet X-ray imaging requirements of any body tissue of the patient during radiotherapy. Therefore, it may be possible to form X-ray images of any site to be treated that meet positioning requirements during radiotherapy (e.g., setup requirements before the radiation therapy and minor adjustments and updates to a treatment plan during radiotherapy), and thus improve an efficiency and accuracy of radiotherapy.

### BRIEF DESCRIPTION OF THE DRAWINGS

In order to describe technical solutions in embodiments of the present disclosure or in the prior art more clearly, the accompanying drawings to be used in the description of the embodiments or in the prior art will be introduced briefly. However, the accompanying drawings to be described below are merely some embodiments of the present disclosure, and a person of ordinary skill in the art can obtain other drawings according to these drawings without paying any creative effort.

FIG. 1 is a schematic structural diagram of a radiotherapy apparatus according to an embodiment of the present disclosure;

FIG. 2 is a schematic structural diagram of an imaging source according to an embodiment of the present disclosure;

FIG. 3 is a schematic structural diagram of another imaging source according to an embodiment of the present disclosure;

FIG. 4 is a schematic structural diagram of another radiotherapy apparatus according to an embodiment of the present disclosure;

FIG. 5 is a schematic structural diagram of yet another radiotherapy apparatus according to an embodiment of the present disclosure;

FIG. 6 is a flow chart of a control method of a radiotherapy apparatus according to an embodiment of the present disclosure;

FIG. 7 is a schematic structural diagram of a processor of a radiotherapy apparatus according to an embodiment of the present disclosure;

FIG. 8 is a schematic structural diagram of a control apparatus of a radiotherapy apparatus according to an embodiment of the present disclosure; and

FIG. 9 is a schematic structural diagram of yet another radiotherapy apparatus according to embodiments of the present disclosure.

### DETAILED DESCRIPTION

The technical solutions in embodiments of the present disclosure will be described clearly and completely with reference to the accompanying drawings in the embodiments of the present disclosure. Obviously, the described embodiments are merely some but not all of embodiments of the present disclosure. All other embodiments obtained by a person of ordinary skill in the art based on the embodiments of the present disclosure without paying any creative effort shall be included in the protection scope of the present disclosure.

It will be noted that, in the embodiments of the present disclosure, words “exemplary” or “such as/for example” are used to indicate an example, an illustration, or a description. Any embodiment or design described with “exemplary” or “such as/for example” in the embodiments of the present disclosure should not be construed as preferred or advantageous over other embodiments or designs. More exactly, the use of the words “exemplary” or “such as/for example” is intended to present the concepts in a particular manner.

It will be noted that, in the embodiments of the present disclosure, “of”, “relevant” and “corresponding” may sometimes be interchanged, and it will be pointed out that in a case where a difference is not emphasized, the three words have the same meaning.

In order to describe the technical solutions provided in embodiments of the present disclosure more clearly, in the embodiments of the present disclosure, words such as “first” and “second” are used to distinguish between identical or similar items with substantially the same function and effect. Those skilled in the art will understand that the words such as “first” and “second” do not limit a quantity or an order of execution.

Existing radiotherapy devices use X-rays of a single energy level for imaging, and can only generate X-ray images of a corresponding energy level. In this case, it is impossible to meet different needs of X-ray imaging of different body tissues of a patient during radiotherapy. As a result, it is impossible to accurately locate a target area using X-ray images during radiotherapy, and an accuracy and effect of radiotherapy are affected.

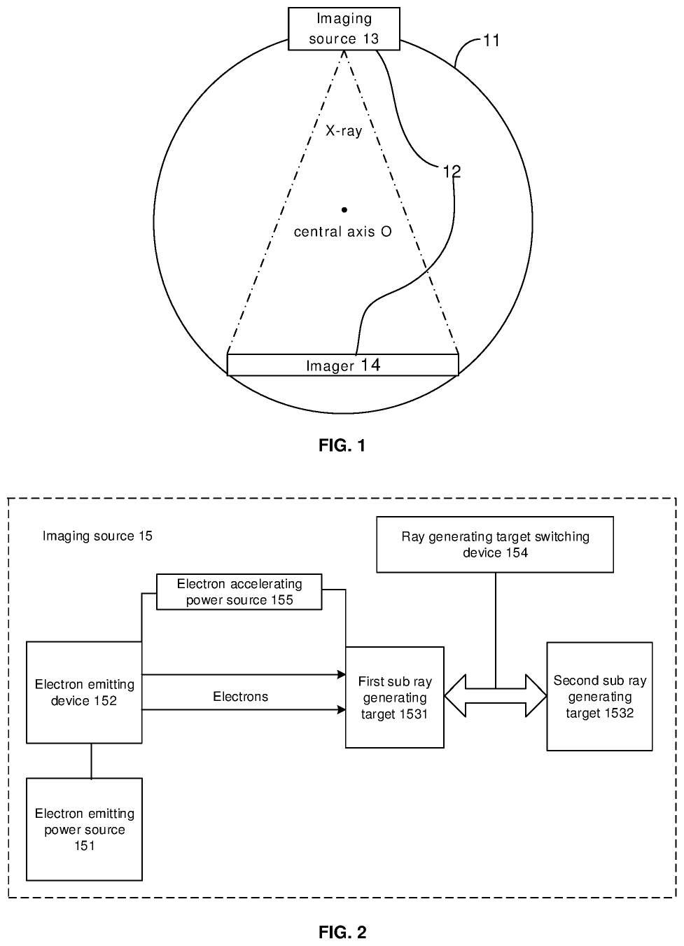

In view of the above problems, referring to FIG. 1, embodiments of the present disclosure provide a radiotherapy apparatus, which includes a rotating gantry 11 rotatable about a central axis O and a multi-energy imaging device 12. The multi-energy imaging device includes an imaging source 13 and an imager 14.

The imaging source 13 and the imager 14 are arranged opposite to each other on the rotating gantry 11. As shown in FIG. 1, in an actual radiotherapy apparatus, the imaging source 13 and the imager 14 provided on the rotating gantry 11 only need to be arranged opposite to each other, so that X-rays emitted by the imaging source 13 can be received by the imager 14.

The imaging source 13 is configured to generate X-rays of at least two energy levels and emit X-rays of at least one energy level in the X-rays of at least two energy levels, so that the X-rays of at least one energy level pass through a site to be treated of the patient. For example, the site to be treated of the patient may be at a central axis O of the rotating gantry. For example, the X-rays of at least two energy levels include at least X-rays of a kilovolt level and X-rays of a megavolt level. The X-rays of at least one energy level is configured to meet imaging requirements of the site to be treated. The X-rays of at least one energy level is configured to meet imaging requirements of the site to be treated.

The imager 14 is configured to receive the X-rays of at least one energy level that pass through the site to be treated, and to generate X-ray images of at least one energy level of the site to be treated according to the X-rays of at least one energy level.

Optionally, the rotating gantry may be a ring gantry or a C-shaped gantry.

With the radiotherapy apparatus provided in the above embodiments, in a case where there is a need to position the site to be treated of the patient, the imager cooperates with the imaging source to generate at least one target X-ray image. Then, the at least one target X-ray image may be registered with at least one pre-stored reference image. Finally, a positional deviation of the site to be treated may be obtained according to the result of registering the at least one target X-ray image with the at least one reference image. After the positional deviation of the site to be treated is obtained, a current position of the site to be treated relative to a treatment bed may be determined according to the positional deviation. Therefore, it may be possible to determine whether the treatment bed should be adjusted to change a position of the patient, so that the radiotherapy goes smoothly. In the technical solutions provided in the embodiments of the present disclosure, the imaging source can generate X-rays of various energy levels. In this way, X-ray images of different energy levels can be generated to meet X-ray imaging requirements of any body tissue of the patient during radiotherapy. Therefore, it may be possible to form X-ray images of any site to be treated that meet positioning requirements during radiotherapy (e.g., setup requirements before the radiation therapy and minor adjustments and updates to a treatment plan during radiotherapy), and thus improve an efficiency and accuracy of radiotherapy.

In order to enable the imaging source in the radiotherapy apparatus provided in the above embodiments to emit X-rays of different energy levels, the embodiments of the present disclosure provide the following four embodiments for illustration.

Embodiment 1: Referring to FIG. 2, an embodiment of the present disclosure provides an imaging source 15 in a radiotherapy apparatus. As for other components in the radiotherapy apparatus, reference can be made to FIG. 1 and corresponding descriptions. The imaging source 15 is a target-switching type imaging source. The target-switching type imaging source is an imaging source that generates X-rays of different energy levels by switching ray generating targets. The ray generating targets are configured to generate X-rays under bombardment of electrons. The imaging source 15 includes: an electron emitting power source 151, an electron emitting device 152, a ray generating target, a ray generating target switching device 154, and an electron accelerating power source 155. The X-rays of at least two energy levels include at least X-rays of a first energy level and X-rays of a second energy level. The ray generating target includes at least two sub ray generating targets, and the at least two sub ray generating targets include at least a first sub ray generating target 1531 and a second sub ray generating target 1532. The electron emitting power source 151 is configured to supply power to the electron emitting device 152, so as to cause the electron emitting device 152 to emit electrons to a preset position. The ray generating target switching device 154 is configured to switch positions of the first sub ray generating target 1531 and the second sub ray generating target 1532, so as to switch sub ray generating targets located at preset position.

The electron accelerating power source 155 is configured to generate an accelerating electric field between the electron emitting device 152 and the preset position, so as to accelerate electrons emitted by the electron emitting device 152.

When the first sub ray generating target 1531 is at the preset position, the first sub ray generating target 1531 is configured to receive the electrons emitted by the electron emitting device 152 and generate the X-rays of the first energy level. When the second sub ray generating target 1532 is at the preset position, the second sub ray generating target 1532 is configured to receive the electrons emitted by the electron emitting device 152 and generate the X-rays of the second energy level.

For example, the first sub ray generating target and the second sub ray generating target in Embodiment 1 may both be composed of tungsten, molybdenum, copper, carbon or alloy, but in different proportions. It will be noted that, according to actual conditions, the imaging source provided in the above embodiment may include multiple sub ray generating targets, so as to generate X-rays of multiple energy levels.

For example, the X-rays of the first energy level in Embodiment 1 are X-rays of a low energy level (50-100 KV), and the X-rays of the second energy level are X-rays of a high energy level (100 KV-6 MV). The composition and number of sub ray generating targets in Embodiment 1 will determine the energy levels of and the types of X-rays that can be provided by the imaging source.

For example, the electron emitting power source 151 in Embodiment 1 is a low voltage power source (5-10V), and the electron emitting device 152 may be composed of any substance that can emit electrons when heated, such as tungsten wire.

For example, the ray generating target switching device 154 in Embodiment 1 may be a drawer type device, and the first sub ray generating target 1531 and the second sub ray generating target 1532 are switched by pushing and pulling. For another example, the ray generating target switching device 154 may be a rotation type device, and the first sub ray generating target 1531 and the second sub ray generating target 1532 are switched by rotation. Any type of the ray generating target switching device 154 may be automatic or manual.

Since there are multiple sub ray generating targets that can be switched from one other in the imaging source 15 provided in Embodiment 1, the imaging source 15 may be able to generate X-rays of multiple energy levels, and thus meet the requirements of radiotherapy apparatuses that require X-rays of multiple energy levels.

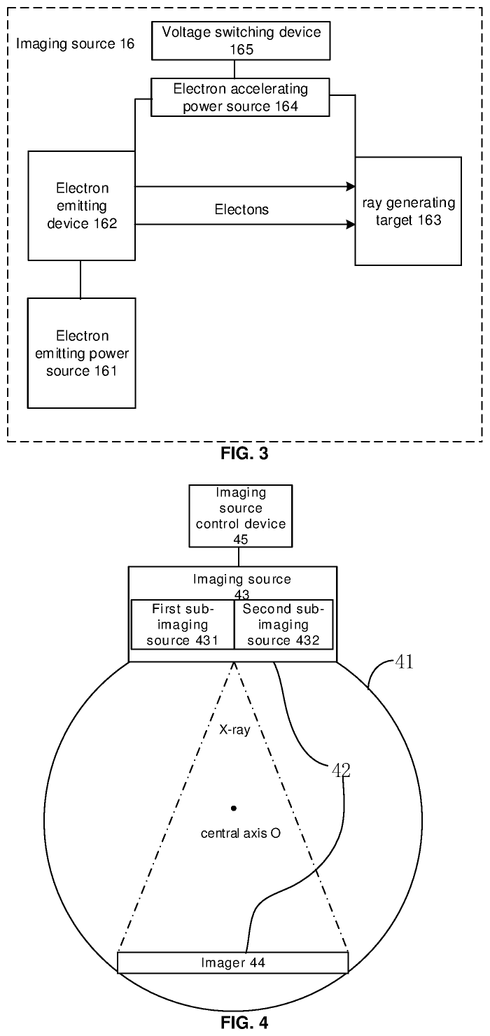

Embodiment 2: Referring to FIG. 3, an embodiment of the present disclosure provides an imaging source 16 in a radiotherapy apparatus. As for other components in the radiotherapy apparatus, reference can be made to FIG. 1 and corresponding descriptions. The imaging source 16 is a voltage-switching type imaging source. The voltage-switching type imaging source is an imaging source that generates X-rays of different energy levels by switching an acceleration voltage of electrons to be accelerated. The electrons to be accelerated are configured to bombard the ray generating target after they are accelerated by the acceleration voltage, so as to cause the ray generating target to generate X-rays. The imaging source 16 includes: an electron emitting power source 161, an electron emitting device 162, a ray generating target 163, an electron accelerating power source 164, and a voltage switching device 165. The electron accelerating power source 164 can provide at least two acceleration voltages, and the at least two acceleration voltages include at least a first acceleration voltage and a second acceleration voltage. The X-rays of at least two energy levels include at least X-rays of a first energy level and X-rays of a second energy level. The electron emitting power source 161 is configured to supply power to the electron emitting device 162, so as to cause the electron emitting device 162 to emit electrons to the ray generating target 163. The electron accelerating power source 164 is configured to use an acceleration voltage supplied by itself to generate an accelerating electric field between the electron emitting device 162 and the ray generating target 163, so as to accelerate electrons emitted by the electron emitting device 162. The voltage switching device 165 is configured to switch acceleration voltages supplied by the electron accelerating power source 164.

When the electron accelerating power source 164 uses the first acceleration voltage to generate the accelerating electric field between the electron emitting device 162 and the ray generating target 163, the ray generating target 163 is configured to receive the electrons emitted by the electron emitting device 162 and generate the X-rays of the first energy level. When the electron accelerating power source 164 uses the second acceleration voltage to generate the accelerating electric field between the electron emitting device 162 and the ray generating target 163, the Ray generating target 163 is configured to receive the electrons emitted by the electron emitting device 162 and generate the X-rays of the second energy level.

For example, in Embodiment 2, there may be only one ray generating target 163. For another example, the ray generating target 163 may include multiple sub ray generating targets, as described in Embodiment 1. In a case where the ray generating target 163 includes the multiple sub ray generating targets, a ray generating target switching device is required. In the case where the ray generating target 163 includes the multiple sub ray generating targets, the imaging source can generate X-rays of m*n energy levels. Herein, m is the number of different voltages that the electron accelerating power source can provide, and n is the number of ray generating targets. Therefore, this embodiment is only an exemplary description, and no specific limitation is imposed on devices other than the electronic accelerating power source.

For example, the electron emitting power source 161 in Embodiment 2 is a low voltage power source (5-10V), and the electron emitting device 162 may be composed of any substance that emits electrons when heated, such as tungsten wire.

For example, the voltage switching device 165 and the electron accelerating power source 164 in Embodiment 2 may together form a variable voltage source (which may be switched automatically or manually), and no specific limitation is imposed on the voltage switching device 165 herein.

For example, the X-rays of the first energy level in Embodiment 2 are X-rays of a low energy level (50-100 KV), and the X-rays of the second energy level are X-rays of a high energy level (100 KV-6 MV). The magnitude and number of voltages that can be provided by the electron accelerating power source 164 in Embodiment 2 will determine the energy levels of and the types of X-rays that can be provided by the imaging source 16.

In the imaging source provided in Embodiment 2, the electron accelerating power source 164 provides different acceleration voltages to change a speed of electrons emitted by the electron emitting device 162 that bombard the ray generating target 163, so that the imaging source can generate X-rays of different energy levels to meet the requirements of the radiotherapy apparatuses that require X-rays of multiple energy levels.

It will be noted that, on the basis of Embodiment 1 and/or Embodiment 2, the ray generating target switching device and/or the voltage switching device may be connected to the rotating gantry of the radiotherapy device (via wired or wireless connection) in actual practice, so that when the rotating gantry of the radiotherapy apparatus shown in FIG. 1 rotates to a preset angle, the energy level of the X-rays generated by the imaging source is changed. The X-rays of at least two energy levels include at least the X-rays of the first energy level and the X-rays of the second energy level.

The preset angle includes at least two preset sub-angles, and the at least two preset sub-angles include at least a first preset sub-angle and a second preset sub-angle. For example, the first preset sub-angle is 0 degrees, and the second preset sub-angle is 180 degrees. However, in a case where there are multiple preset sub-angles, the multiple preset sub-angles may be set according to the actual situation, and are not limited herein. The imaging source generates the X-rays of the first energy level when the rotating gantry rotates to the first preset sub-angle; and the imaging source generates the X-rays of the second energy level when the rotating gantry rotates to the second preset sub-angle. For example, when the rotating gantry rotates to 0 degrees, the imaging source generates the X-rays of the first energy level; when the rotating gantry rotates to 180 degrees (that is, when the rotating gantry rotates to 180 degrees), the imaging source generates the X-rays of the second energy level; when the rotating gantry rotates to another 180 degrees (that is, when the rotating gantry rotates to 0 degrees again), the imaging source generates the X-rays of the first energy level; and so forth. The first preset sub-angle and the second preset sub-angle may not be identical, which is not limited here.

Embodiment 3: Referring to FIG. 4, an embodiment of the present disclosure provides a radiotherapy apparatus, which includes: a rotating gantry 41 rotatable about a central axis O, a multi-energy imaging device 42, and an imaging source control device 45. The multi-energy imaging device 42 includes an imaging source 43 and an imager 44. The imaging source 43 and the imager 44 are arranged opposite to each other on the rotating gantry 41. As shown in FIG. 4, in an actual radiotherapy apparatus, the imaging source 43 and the imager 44 provided on the rotating gantry 41 only need to be arranged opposite to each other, so that the X-rays emitted by the imaging source 43 can be received by the imager 44. The imaging source 43 includes at least two sub-imaging sources, and the at least two sub-imaging sources include at least a first sub-imaging source 431 and a second sub-imaging source 432. The imaging source 43 is configured to generate X-rays of at least two energy levels, and the X-rays of at least two energy levels include at least X-rays of a first energy level and X-rays of a second energy level. The first sub-imaging source 431 is configured to generate X-rays of the first energy level, and the second sub-imaging source 432 is configured to generate X-rays of the second energy level. The imaging source control device 45 is configured to control switching of the sub-imaging sources that emit X-rays, which are to pass through the site to be treated of the patient. The imager 44 is configured to receive X-rays of target energy levels that pass through the site to be treated, and to generate X-ray images of target energy levels of the site to be treated according to the X-rays of target energy levels.

It will be noted that, when the imaging source control device 45 controls the sub-imaging sources to be switched, the number of sub-imaging sources controlled by the imaging source control device is not limited. In a case where only one sub-imaging source in the radiotherapy apparatus emits X-rays at a same moment, the imaging source control device 45 may switch only one sub-imaging source each time. In a case where multiple sub-imaging sources in the radiotherapy apparatus emit X-rays at the same moment, the imaging source control device may switch one or more sub-imaging sources each time. A specific configuration depends on the actual situation.

For example, in Embodiment 3, the X-rays of the first energy level are X-rays of a low energy level (50-100 KV), and the X-rays of the second energy level are X-rays of a high energy level (100 KV-6 MV). The number and types of sub-imaging sources in Embodiment 3 will determine the energy levels of and the types of X-rays that can be provided by the imaging source.

For example, the imaging source control device 45 in Embodiment 3 is a mechanical control device. For another example, the imaging source control device 45 in Embodiment 3 is a software control device. In a case where the imaging source control device 45 is the mechanical control device, the imaging source control device 45 may be an independent device; or, the rotating gantry may be reused as the imaging source control device. In this case, the sub-imaging sources may be switched when the rotating gantry rotates by a certain angle.

It will be noted that, as for respective structures of the sub-imaging sources in Embodiment 3, reference can be made to the descriptions of Embodiment 1 and Embodiment 2, which can be freely combined. Details will not be repeated here, as long as it is ensured that the two sub-imaging sources can generate X-rays of different energy levels.

In the radiotherapy apparatus provided in Embodiment 3, different sub-imaging sources that can generate X-rays of different energy levels are provided. In this way, the radiotherapy apparatus can use X-rays of different energy levels to irradiate the site to be treated of the patient during operation, so as to obtain X-ray images of different energy levels. Therefore, the positional deviation can be obtained after the obtained X-ray images are registered with the at least one pre-stored reference image. Then, the position of the patient or the treatment plan can be adjusted according to the positional deviation, thereby improving the efficiency and effect of radiotherapy.

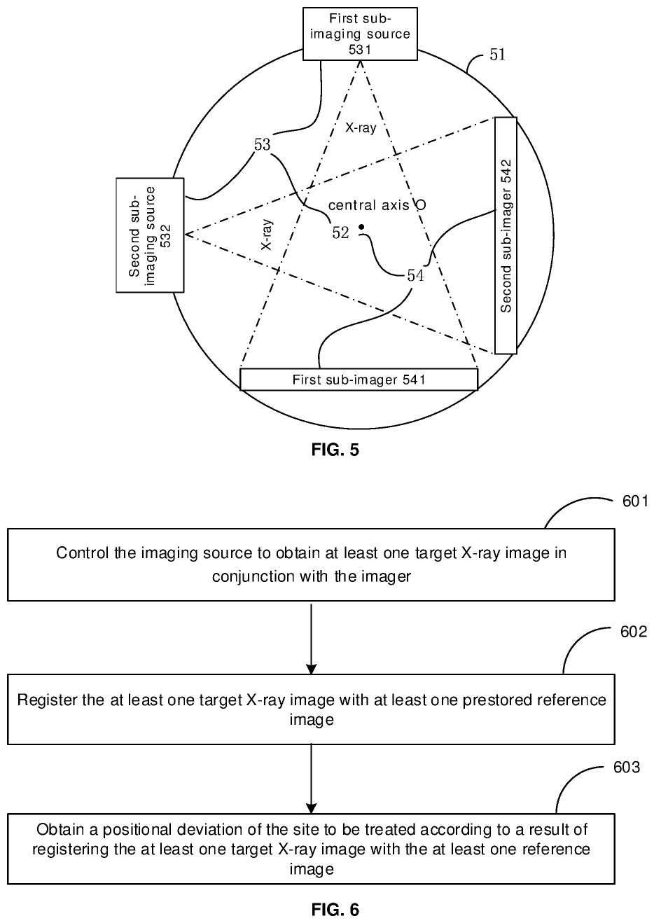

Embodiment 4: In order to save more time in the radiotherapy process and improve the efficiency of radiotherapy, referring to FIG. 5, an embodiment of the present disclosure provides a radiotherapy apparatus, which includes: a rotating gantry 51 rotatable about a central axis O and a multi-energy imaging device 52. The multi-energy imaging device 52 includes an imaging source 53 and an imager 54. The imaging source 53 includes at least two sub-imaging sources, and the at least two sub-imaging sources includes at least a first sub-imaging source 531 and a second sub-imaging source 532. The imager 54 includes at least two sub-imagers, and the at least two sub-imagers include at least a first sub-imager 541 and a second sub-imager 542. The imaging source 53 is configured to generate X-rays of at least two energy levels, and the X-rays of at least two energy levels include at least X-rays of a first energy level and X-rays of a second energy level. The first sub-imaging source 531 is configured to generate X-rays of the first energy level, and the second sub-imaging source 432 is configured to generate X-rays of the second energy level. The first sub-imaging source 531 and the first sub-imager 541 are arranged opposite to each other on the rotating gantry 51, and the second sub-imaging source 532 and the second sub-imager 542 are arranged opposite to each other on the rotating gantry 51. A line connecting a position of the first sub-imaging source 531 on the rotating gantry 51 and a position of the first sub-imager 541 on the rotating gantry 51 intersects a line connecting a position of the second sub-imaging source 532 on the rotating gantry 51 and a position of the second sub-imager 542 on the rotating gantry 51. For example, the two lines may intersect at a right angle, and such imaging method is called orthogonal dual-plate imaging in actual practice. The first sub-imaging source 531 is configured to generate the X-rays of the first energy level and emit the X-rays of the first energy level, which are to pass through the site to be treated and reach the first sub-imager 541, so that the first sub-imager 541 generates X-ray images of the first energy level of the site to be treated according to the X-rays of the first energy level. The second sub-imaging source 532 is configured to generate the X-rays of the second energy level and emit the X-rays of the second energy level, which are to pass through the site to be treated and reach the second sub-imager 542, so that the second sub-imager 542 generates X-ray images of the second energy level of the site to be treated according to the X-rays of the second energy level.

It will be noted that, with regard to configurations of the imaging source 53 and the rotating gantry 51 in Embodiment 4, reference can be made to the various configurations in Embodiment 1, Embodiment 2, and Embodiment 3. The four embodiments can be freely combined, and no specific limitation is imposed here.

For example, in Embodiment 4, the X-rays of the first energy level are X-rays of a low energy level (50-100 KV), and the X-rays of the second energy level are X-rays of a high energy level (100 KV-6 MV). The number and types of sub-imaging sources in Embodiment 4 will determine the energy levels of and the types of X-rays that can be provided by the imaging source.

In the radiotherapy apparatus provided in Embodiment 4, various pairs of sub-imaging sources and sub-imagers arranged opposite to each other are provided on the rotatable gantry 51. In this way, the radiotherapy apparatus may be able to simultaneously generate X-ray images corresponding to X-rays of multiple energy levels when X-ray imaging is needed. Compared with the technical solutions provided in Embodiment 1, Embodiment 2 and Embodiment 3, the radiotherapy apparatus provided in Embodiment 4 may be more efficient in obtaining X-ray images of different energy levels, and may be able to increase the efficiency of radiotherapy more significantly.

For example, the imager in the above embodiments includes at least the first sub-imager and the second sub-imager. The X-rays of at least two energy levels include at least the X-rays of the first energy level and the X-rays of the second energy level. The first sub-imager is configured to receive the X-rays of the first energy level that pass through the site to be treated, and to generate X-ray images of the first energy level of the site to be treated according to the X-rays of the first energy level. The second sub-imager is configured to receive the X-rays of the second energy level that pass through the site to be treated, and to generate X-ray images of the second energy level of the site to be treated according to the X-rays of the second energy level.

The first sub-imager receives the X-rays of the first energy level and deposits energy to release visible light, and then converts light signals into electrical signals. X-rays of the second energy level that do not interact with the first sub-imager are absorbed by the second sub-imager, and then visible light is released, and light signals are converted into electrical signals. In this way, X-ray images of the first energy level and X-ray images of the second energy level are generated separately.

In summary, the radiotherapy apparatus provided in the embodiments of the present disclosure includes a rotating gantry rotatable about a central axis O and a multi-energy imaging device. The multi-energy imaging device includes an imaging source and an imager. The imaging source and the imager are arranged opposite to each other on the rotating gantry. The imaging source is configured to generate X-rays of at least two energy levels and emit X-rays of at least one energy level in the X-rays of at least two energy levels, so that the X-rays of at least one energy level pass through the site to be treated of the patient. The imager is configured to receive the X-rays of at least one energy level that pass through the site to be treated, and to generate X-ray images of at least one energy level of the site to be treated according to the X-rays of at least one energy level. Therefore, in a case where there is a need to position the site to be treated of the patient, the imager cooperates with the imaging source to generate at least one target X-ray image. Then, the at least one target X-ray image may be registered with the at least one pre-stored reference image. Finally, the positional deviation of the site to be treated may be obtained according to the result of registering the at least one target X-ray image with the at least one reference image. After the positional deviation of the site to be treated is obtained, the current position of the site to be treated relative to the treatment bed may be determined according to the positional deviation. Therefore, it may be possible to determine whether the treatment bed should be adjusted to change the position of the patient, so that the radiotherapy goes smoothly. In the technical solutions provided in the embodiments of the present disclosure, the imaging source can generate X-rays of various energy levels. In this way, the imager can generate X-ray images of different energy levels to meet X-ray imaging requirements of any body tissue of the patient during radiotherapy. Therefore, it may be possible to form X-ray images of any site to be treated that meet positioning requirements during radiotherapy (e.g., setup requirements before the radiation therapy and minor adjustments and updates to the treatment plan during radiotherapy), and thus improve the efficiency and accuracy of radiotherapy.

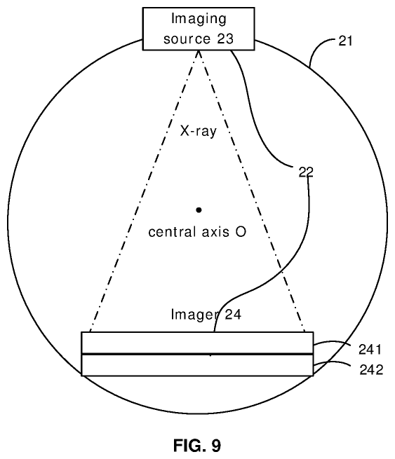

Referring to FIG. 9, some embodiments of the present disclosure further provide a radiotherapy apparatus, which includes a rotating gantry 21 rotatable about a central axis O, a multi-energy imaging device 22 and at least one processor disposed on the rotating gantry 21. The multi-energy imaging device 22 includes an imaging source 23 and an imager 24. The at least one processor is configured to control the imaging source 23 to obtain at least one target X-ray image in conjunction with the imager 24. The at least one target X-ray image is at least one image of X-ray images of at least one energy level of the site to be treated. The at least one target X-ray image is registered with at least one pre-stored reference image. The positional deviation of the site to be treated may be obtained according to the result of registering the at least one target X-ray image with the at least one reference image.

In this way, the radiotherapy apparatus may meet X-ray imaging requirements of any site to be treated of the patient, so as to meet positioning requirements during radiotherapy (e.g., setup requirements before the radiation therapy and minor adjustments and updates to a treatment plan during radiotherapy), and thus improve an efficiency and accuracy of radiotherapy.

In some embodiments, the imaging source 23 is configured to generate X-rays and emit the X-rays, so that the X-rays pass through the site to be treated of the patient. The imager 24 is a multi-layer flat plate detector, and is configured to receive the X-rays that pass through the site to be treated and to generate X-ray images of at least two energy levels of the site to be treated.

In some examples, the multi-layer flat plate detector includes multiple detection layers, and the multiple detection layers are stacked in a direction of a radiation path of the X-rays. The multiple detection layers may identify and absorb X-ray photons according to their energy levels, thereby generating X-ray images of multiple energy levels.

For example, referring to FIG. 9, the multi-layer flat plate detector is a dual-layer flat plate detector. The two-layer flat panel detector includes a top detection layer 241 and a bottom detection layer 242 that are stacked. The top detection layer 241 is disposed adjacent to the imaging source 23, and the bottom detection layer 242 is disposed at a side of the top detection layer 241 away from the imaging source 23. The top detection layer 241 is configured to absorb low energy X-rays and allow high energy X-rays to pass. The bottom detection layer 242 is configured to absorb high energy X-rays. In this way, after the imaging source 23 emits X-rays, the imager 24 receives the X-rays that pass through the site to be treated, and classifies energy levels of the X-rays into two different energy levels according to the magnitude of the energy level of the X-rays, i.e., the low energy level and the high energy level. The high energy X-rays and the low energy X-rays are absorbed by different detection layers to generate X-ray images of the two energy levels. Thereby, high energy data and low energy data of X-rays may be analyzed and split during one scan process, for example during a computed tomography (CT) scan process, which improves the scan efficiency.

In some embodiments, the imaging source is configured to generate X-rays of at least two energy levels, and emit X-rays of at least one energy level in the X-rays of at least two energy levels to pass through the site to be treated of the patient. The X-rays of at least one energy level are configured to meet imaging requirements of the site to be treated. The imager is configured to receive the X-rays of at least one energy level that pass through the site to be treated, and to generate at least one X-ray image of at least one energy level of the site to be treated according to the X-rays of at least one energy level.

In some examples, the imaging source 43 includes at least two sub-imaging sources, and the at least two sub-imaging sources include a first sub-imaging source 431 and a second sub-imaging source 432. The imaging source 43 is configured to generate X-rays of at least two energy levels. The X-rays of at least two energy levels include X-rays of a first energy level and X-rays of a second energy level. The first sub-imaging source 431 is configured to generate the X-rays of the first energy level, and the second sub-imaging source 432 is configured to generate the X-rays of the second energy level. On this basis, the radiotherapy apparatus further includes an imaging source control device 45. The imaging source control device 45 is configured to switch the sub-imaging sources that emit X-rays passing through the site to be treated of the patient.

In some other examples, the imager includes at least a first sub-imager and a second sub-imager. The X-rays of at least two energy levels include at least the X-rays of the first energy level and the X-rays of the second energy level. The first sub-imager is configured to receive the X-rays of the first energy level that pass through the site to be treated, and to generate X-ray images of the first energy level of the site to be treated according to the X-rays of the first energy level. The second sub-imager is configured to receive the X-rays of the second energy level that pass through the site to be treated, and to generate X-ray images of the second energy level of the site to be treated according to the X-rays of the second energy level.

The first sub-imager receives the X-rays of the first energy level and deposits energy to release visible light, and then converts light signals into electrical signals. X-rays of the second energy level that do not interact with the first sub-imager are absorbed by the second sub-imager, and then visible light is released, and light signals are converted into electrical signals. In this way, X-ray images of the first energy level and X-ray images of the second energy level are generated separately.

In some other examples, the imaging source 53 includes at least two sub-imaging sources, and the at least two sub-imaging sources includes at least a first sub-imaging source 531 and a second sub-imaging source 532. The imager 54 includes at least two sub-imagers, and the at least two sub-imagers include at least a first sub-imager 541 and a second sub-imager 542. The first sub-imaging source 531 and the first sub-imager 541 are arranged opposite to each other on the rotating gantry 51, and the second sub-imaging source 532 and the second sub-imager 542 are arranged opposite to each other on the rotating gantry 51. A line connecting a position of the first sub-imaging source 531 on the rotating gantry 51 and a position of the first sub-imager 541 on the rotating gantry 51 intersects a line connecting a position of the second sub-imaging source 532 on the rotating gantry 51 and a position of the second sub-imager 542 on the rotating gantry 51. The first sub-imaging source 531 is configured to generate the X-rays of the first energy level and emit the X-rays of the first energy level, which are to pass through the site to be treated and reach the first sub-imager 541, so that the first sub-imager 541 generates X-ray images of the first energy level of the site to be treated according to the X-rays of the first energy level. The second sub-imaging source 532 is configured to generate the X-rays of the second energy level and emit the X-rays of the second energy level, which are to pass through the site to be treated and reach the second sub-imager 542, so that the second sub-imager 542 generates X-ray images of the second energy level of the site to be treated according to the X-rays of the second energy level.

In addition, with regard to configurations of the rotating gantry 21, the imaging source 23 and the imager 24, reference can be made to the various configurations in Embodiment 1, Embodiment 2, Embodiment 3 and Embodiment 4, and various configurations based on these embodiments, which are not described herein again.

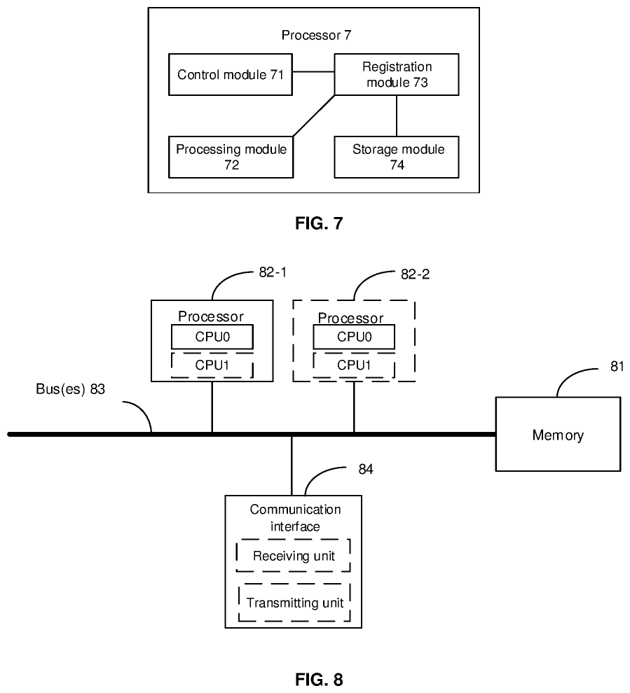

In some examples, referring to FIG. 7, a processor 7 includes a control module 71, a processing module 72, a registration module 73 and a storage module 74. The control module 71 is configured to control the imaging source to obtain at least one target X-ray image in conjunction with the imager. The registration module 73 is configured to register the at least one target X-ray image obtained by the control module 71 with at least one reference image pre-stored in the storage module 74. The processing module 72 is configured to obtain a positional deviation of the site to be treated according to a result of registering the at least one target X-ray image with the at least one reference image by the registration module 73.

For example, the control module 71 is configured to control the imaging source to emit the X-rays of at least two energy levels to pass through the site to be treated of the patient, so that the imager generates X-ray images of at least two energy levels according to the X-rays of at least two energy levels that pass through the site to be treated; and select at least one target X-ray image from the X-ray images of at least two energy levels according to information of the site to be treated pre-stored in the storage module 74.

For another example, the control module 71 is configured to control the imaging source to emit X-rays of at least two target energy levels to pass through the site to be treated of the patient, so that the imager generates X-ray images of at least two target energy levels according to the X-rays of at least two target energy levels that pass through the site to be treated; and select at least one target X-ray image from the X-ray images of at least two energy levels according to the information of the site to be treated pre-stored in the storage module 74.

For yet another example, the control module 71 is configured to control the imaging source to emit X-rays to pass through the site to be treated of the patient, so that the imager generates X-ray images of at least two energy levels according to the X-rays that pass through the site to be treated; and select at least one target X-ray image from the X-ray images of at least two energy levels according to the information of the site to be treated pre-stored in the storage module 74.

For example, when the control module 71 controls the imaging source to emit preset X-rays to pass through the site to be treated of the patient, the control module 71 is further configured to control the rotating gantry to rotate to at least two different imaging angles, so that there are at least two X-ray images respectively corresponding to different imaging angles in X-ray images generated by the imager according to X-rays of each energy level. It will be noted that the preset X-rays are the X-rays of at least two energy levels or the X-rays of the target energy level.

For example, before the radiotherapy apparatus is used in treating the patient, the positional deviation includes a setup error. After the processing module 72 obtains the positional deviation, the processing module 72 is further configured to control the radiotherapy apparatus to adjust a position of the patient according to the positional deviation. When the radiotherapy apparatus is being used in treating the patient, the positional deviation includes a positional deviation of a tumor in the site to be treated. After the processing module 72 obtains the positional deviation, the processing module 72 is further configured to determine whether the positional deviation of the tumor is within a preset deviation range; and perform deviation correction according to the at least one target X-ray image in response to the positional deviation of the tumor being not within the preset deviation range. The deviation correction may refer to adjusting the position of the patient, adjusting the treatment area (e.g., an accelerator), or updating the treatment plan pre-stored in the storage module 74.

The registration module 73 is configured to process different target X-ray images in the target X-ray images obtained by the control module 71; and register at least one processed target X-ray image with the at least one reference image pre-stored in the storage module 74.

For example, in a case where the target X-ray image obtained by the control module 71 and the reference image stored in the storage module 74 are both three-dimensional images, the registration module 73 is configured to register a sagittal plane, a coronal plane and a transverse plane of the target X-ray image obtained by the control module 71 with a sagittal plane, a coronal plane and a transverse plane of the reference image pre-stored in the storage module 74 respectively.

Referring to FIG. 6, embodiments of the present disclosure further provide a control method of a radiotherapy apparatus, which includes steps 601 to 603.

In 601, the imaging source is controlled to obtain at least one target X-ray image in conjunction with the imager.

Herein, the at least one target X-ray image may be at least one of X-ray images of at least one energy level of the site to be treated.

For example, that the imaging source is controlled to obtain the at least one target X-ray image in conjunction with the imager may include the following steps. The imaging source may be controlled to emit the X-rays of at least two energy levels, which are to pass through the site to be treated, so that the imager generates X-ray images of at least two energy levels, and then at least one desired target X-ray image is selected from the X-ray images of at least two energy levels according to information of the site to be treated. For another example, that the imaging source is controlled to obtain the at least one target X-ray image in conjunction with the imager may include the following steps. The at least one desired energy level of X-rays that need to be emitted by the emitting source may be set as the target energy level according to the information of the site to be treated first, and then the imaging source is controlled to emit the X-rays of the target energy level which are to pass through the site to be treated, so that the imager generates the at least one target X-ray image. For yet another example, that the imaging source is controlled to obtain the at least one target X-ray image in conjunction with the imager may include the following steps. The imaging source may be controlled to emit the X-rays, which are to pass through the site to be treated, so that the imager generates X-ray images of at least two energy levels, and then at least one desired target X-ray image is selected from the X-ray images of at least two energy levels according to information of the site to be treated.

In addition, in order to ensure an accuracy of the result of registering the at least one target X-ray image with the at least one reference image in a subsequent process, there is a need to obtain X-ray images of each energy level (or the target energy level) at at least two imaging angles. Therefore, when the imaging source is controlled to emit at least preset X-rays (the preset X-rays are the X-rays of at least two energy levels or the X-rays of the target energy level), which are to pass through the site to be treated of the patient, the control method further includes: controlling the rotating gantry to rotate to at least two different imaging angles, so that there are at least two X-ray images respectively corresponding to different imaging angles in X-ray images generated by the imager according to X-rays of each energy level (or the target energy level).

In 602, the at least one target X-ray image is registered with at least one pre-stored reference image.

It will be noted that, in a case where the target X-ray image is a two-dimensional image, the reference image is generally a DRR (Digitally Reconstructed Radiograph) image generated from a CT (Computed Tomography) image. However, in a case where the reference image (a DRR image, a CT image, or other images) is a three-dimensional image, in actual practice, there is one more step in step 602—an image reconstruction step, so that the obtained target X-ray image is reconstructed into a three-dimensional image. Then, a sagittal plane, a coronal plane and a transverse plane of the reconstructed target X-ray image are registered with a sagittal plane, a coronal plane and a transverse plane of the reference image respectively. For example, since there may be multiple images in the obtained target X-ray images in actual practice, and a clearest part of each image is different, in order to ensure a more accurate registration result, step 602 includes steps 6021 to 6022.

In 6021, different target X-ray images in the target X-ray images are processed.

For example, the description that the target X-ray images are processed means that different target X-ray images are merged and reconstructed to obtain a reconstructed target X-ray image. In the reconstruction process, the number of dimensions of the target X-ray image may be or may not be changed, which depends on the actual occasion.

In 6022, the at least one processed target X-ray image is registered with the at least one reference image. In 603, a positional deviation of the site to be treated is obtained according to a result of registering the at least one target X-ray image with the at least one reference image.

Optionally, before the radiotherapy apparatus is used in treating the patient, the site to be treated of the patient needs to be fixed during set-up. In this case, the positional deviation obtained in the embodiments of the present disclosure includes a setup error, and the control method further includes step 604.

In 604, the radiotherapy apparatus is controlled to adjust a position of the patient according to the positional deviation.

For example, the treatment bed may be adjusted to adjust the position of the patient, or other devices may be adjusted to adjust the position of the patient.

Optionally, when the radiotherapy apparatus is being used in treating the patient, since a position of a tumor in the site to be treated of the patient may change with the physiological or inadvertent activity of the patient, the positional deviation obtained in the embodiments of the present disclosure includes a positional deviation of the tumor in the site to be treated, and the control method further includes step 605.

In 605, it is determined whether the positional deviation of the tumor is within a preset deviation range.

Step 60422 is performed in response to the positional deviation of the tumor being not within the preset deviation range. The preset deviation range is a deviation range relative to 0, for example, [−0.1 mm, +0.1 mm].

In 60422, deviation correction is performed according to the at least one target X-ray image.

For example, the deviation correction may refer to adjusting the position of the patient, adjusting the treatment area (e.g., an accelerator), or updating the pre-stored treatment plan. Herein, the treatment plan may include movements of the treatment bed during radiotherapy and specific moments when the energy levels of the X-rays in the radiotherapy apparatus are switched during the treatment process.

In the control method of the radiotherapy apparatus provided in the embodiments of the present disclosure, in a case where there is a need to position the site to be treated of the patient, it may be possible to control the imaging source to generate at least one target X-ray image in conjunction with the imager. Then, the at least one target X-ray image may be registered with the at least one pre-stored reference image. Finally, the positional deviation of the site to be treated may be obtained according to the result of registering the at least target X-ray image with the at least one reference image. After the positional deviation of the site to be treated is obtained, the current position of the site to be treated relative to the treatment bed may be determined according to the positional deviation. Therefore, it may be possible to determine whether the treatment bed should be adjusted to change the position of the patient, so that the radiotherapy goes smoothly. In the technical solutions provided in the embodiments of the present disclosure, the imaging source can generate X-rays of various energy levels. In this way, X-ray images of different energy levels can be generated to meet X-ray imaging requirements of any body tissue of the patient during radiotherapy. Therefore, it may be possible to form X-ray images of any site to be treated that meet positioning requirements during radiotherapy (e.g., setup requirements before the radiation therapy and minor adjustments and updates to the treatment plan during radiotherapy), and thus improve the efficiency and accuracy of radiotherapy.

Referring to FIG. 8, embodiments of the present disclosure further provide another control apparatus of the radiotherapy apparatus, which includes: a memory 81, at least one processor 82 (82-1 and 82-2), at least one bus 83, and a communication interface 84. The memory 81 is configured to store computer-executable instructions, and the at least one processor 82 and the memory 81 are connected through the at least one bus 83. When the control apparatus of the radiotherapy apparatus is running, the at least one processor 82 executes the computer-executable instructions stored in the memory 81, so that the control apparatus of the radiotherapy apparatus performs the control method of the radiotherapy apparatus provided in the above embodiments.

In a specific implementation, as an embodiment, the at least one processor 82 (82-1 and 82-2) may include one or more CPUs, such as CPU0 and CPU1 shown in FIG. 8. Moreover, as an embodiment, the control apparatus of the radiotherapy apparatus may include multiple processors 82, such as the processor 82-1 and processor 82-2 shown in FIG. 8. Each of the processors 82 may be a single core processor (Single-CPU) or a multi-core processor (Multi-CPU). The at least one processor 82 herein may refer to one or more devices, circuits, and/or processing cores for processing data (such as computer program instructions).

The memory 81 may be a read-only memory (ROM) or other types of static storage devices that can store static information and instructions, a random access memory (RAM) or other types of dynamic storage devices that can store information and instructions, an electrically erasable programmable read-only memory (EEPROM), a compact disc read-only memory (CD-ROM) or other optical disk storage medium, optical disc storage medium (including compact discs, laser discs, CDs, digital universal discs, blu-ray discs, etc.), disk storage medium or other magnetic storage devices, or any other medium that can be used to carry or store desired program codes in the form of instructions or data structures that can be accessed by a computer, which is not limited thereto. The memory 81 may be an independent device, which is connected to the at least one processor 82 through a communication bus 83. The memory 81 may also be integrated with the at least one processor 82.

In a specific implementation, the memory 81 is configured to store data in the present disclosure and computer-executable instructions corresponding to software programs executing the present disclosure. The at least one processor 82 may execute various functions of the control apparatus of the radiotherapy apparatus by running or executing the software programs stored in the memory 81 and calling the data stored in the memory 81.

The communication interface 84 is any device like a transceiver, and is configured to communicate with other devices or communication networks, such as control systems, radio access networks (RAN), and wireless local area networks (WLAN). The communication interface 84 may include a receiving unit to realize a receiving function, and a transmitting unit to realize a transmitting function.

The at least one bus 83 may be an industry standard architecture (ISA) bus, a peripheral component interconnect (PCI) bus, or an extended industry standard architecture (EISA) bus. The at least one bus 83 can be divided into an address bus, a data bus, a control bus, etc. For ease of illustration, the at least one bus 83 is indicated by only one thick line in FIG. 8. However, it does not mean that there is only one bus or one type of bus.

In summary, in the radiotherapy apparatus and the control method and control apparatus thereof provided in the embodiments of the present disclosure, the radiotherapy apparatus includes a rotating gantry rotatable about a central axis and a multi-energy imaging device. The multi-energy imaging device includes an imaging source and an imager. The imaging source and the imager are arranged opposite to each other on the rotating gantry. The imaging source is configured to generate X-rays of at least two energy levels and emit X-rays of at least one energy level in the X-rays of at least two energy levels to pass through the site to be treated of the patient. The imager is configured to receive the X-rays of at least one energy level that pass through the site to be treated and generate X-ray images of at least one energy level of the site to be treated according to the X-rays of at least one energy level. Therefore, in a case where there is a need to position the site to be treated of the patient, it may be possible to control the imaging source to generate at least one target X-ray image in conjunction with the imager. Then, the at least one target X-ray image may be registered with the at least one pre-stored reference image. Finally, the positional deviation of the site to be treated may be obtained according to the result of registering the at least one target X-ray image with the at least one reference image. After the positional deviation of the site to be treated is obtained, the current position of the site to be treated relative to the treatment bed may be determined according to the positional deviation. Therefore, it may be possible to determine whether the treatment bed should be adjusted to change the position of the patient, so that the radiotherapy goes smoothly. In the technical solutions provided in the embodiments of the present disclosure, the imaging source can generate X-rays of various energy levels. In this way, X-ray images of different energy levels can be generated to meet X-ray imaging requirements of any body tissue of the patient during radiotherapy. Therefore, it may be possible to form X-ray images of any site to be treated that meet positioning requirements during radiotherapy (e.g., setup requirements before the radiation therapy and minor adjustments and updates to the treatment plan during radiotherapy), and thus improve the efficiency and accuracy of radiotherapy.

Embodiments of the present disclosure further provide a computer program, which can be directly loaded into a memory and contains software codes. The computer program can implement the control method of the radiotherapy apparatus described above after it is loaded into and executed by a computer.

A person skilled in the art will appreciate that in one or more of the examples described above, the functions described in the present disclosure may be implemented by using a hardware, a software, a firmware, or any combination thereof. When implemented in software, the functions may be stored on a computer-readable medium or transmitted as one or more instructions or codes on a computer-readable medium. The computer-readable medium includes a computer storage medium and a communication medium, and the communication medium includes any medium convenient for transmitting computer programs from one location to another. The storage medium may be any available medium that can be accessed by a general-purpose or special-purpose computer.

The foregoing descriptions are merely some specific implementation manners of the present disclosure, but the protection scope of the present disclosure is not limited thereto. Any person skilled in the art could readily conceive of changes or replacements within the technical scope disclosed by the present disclosure, which shall all be included in the protection scope of the present disclosure. Therefore, the protection scope of the present disclosure shall be subject to the protection scope of the claims.

What is claimed is:

1. A radiotherapy apparatus, comprising:
a rotating gantry rotatable about a central axis; and
a multi-energy imaging device, wherein the multi-energy imaging device includes:
an imaging source configured to generate X-rays of at least two energy levels and emit X-rays of at least one energy level in the X-rays of at least two energy levels, so that the X-rays of at least one energy level pass through a site to be treated of a patient, wherein the X-ray of at least one energy level is configured to meet imaging requirements of the site to be treated; and
an imager configured to receive the X-rays of at least one energy level that pass through the site to be treated, and to generate X-ray images of at least one energy level of the site to be treated according to the X-rays of at least one energy level, wherein
the imaging source and the imager are arranged opposite to each other on the rotating gantry.

2. The radiotherapy apparatus according to claim 1, wherein the imaging source includes at least one of a target-switching type imaging source and a voltage-switching type imaging source;
the target-switching type imaging source is configured to generate X-rays of different energy levels by switching ray generating targets; and
the voltage-switching type imaging source is configured to generate X-rays of different energy levels by switching an acceleration voltage of electrons to be accelerated.

3. The radiotherapy apparatus according to claim 1, wherein the imaging source includes at least two sub-imaging sources, and the at least two sub-imaging sources include at least a first sub-imaging source and a second sub-imaging source;
the X-rays of at least two energy levels include at least X-rays of a first energy level and X-rays of a second energy level;
the first sub-imaging source is configured to generate the X-rays of the first energy level, and the second sub-imaging source is configured to generate the X-rays of the second energy level; and
the radiotherapy apparatus further comprises an imaging source control device configured to control switching of sub-imaging sources that emit X-rays, which are to pass through the site to be treated of the patient.

4. The radiotherapy apparatus according to claim 1, wherein the imaging source includes at least two sub-imaging sources, and the at least two sub-imaging sources include at least a first sub-imaging source and a second sub-imaging source; the imager includes at least two sub-imagers, and the at least two sub-imagers include at least a first sub-imager and a second sub-imager;
the X-rays of at least two energy levels include at least X-rays of a first energy level and X-rays of a second energy level;
the first sub-imaging source and the first sub-imager are arranged opposite to each other on the rotating gantry, and the second sub-imaging source and the second sub-imager are arranged opposite to each other on the rotating gantry;
a line connecting a position of the first sub-imaging source on the rotating gantry and a position of the first sub-imager on the rotating gantry intersects a line connecting a position of the second sub-imaging source on the rotating gantry and a position of the second sub-imager on the rotating gantry;
the first sub-imaging source is configured to generate the X-rays of the first energy level and emit the X-rays of the first energy level, which are to pass through the site to be treated and then reach the first sub-imager, so that the first sub-imager generates X-ray images of the first energy level of the site to be treated according to the X-rays of the first energy level; and
the second sub-imaging source is configured to generate the X-rays of the second energy level and emit the X-rays of the second energy level, which are to pass through the site to be treated and then reach the second sub-imager, so that the second sub-imager generates X-ray images of the second energy level of the site to be treated according to the X-rays of the second energy level.

5. The radiotherapy apparatus according to claim 1, wherein the X-rays of at least two energy levels include at least X-rays of a first energy level and X-rays of a second energy level;
X-rays generated by the imaging source are changed when the rotating gantry rotates to a preset angle;
the preset angle includes at least two preset sub-angles, and the at least two preset sub-angles include at least a first preset sub-angle and a second preset sub-angle;
the imaging source generates the X-rays of the first energy level when the rotating gantry rotates to the first preset sub-angle; and
the imaging source generates the X-rays of the second energy level when the rotating gantry rotates to the second preset sub-angle.

6. The radiotherapy apparatus according to claim 1, wherein the imager includes at least a first sub-imager and a second sub-imager;
the X-rays of at least two energy levels include at least X-rays of a first energy level and X-rays of a second energy level;
the first sub-imager is configured to receive the X-rays of the first energy level that pass through the site to be treated, and to generate X-ray images of the first energy level of the site to be treated according to the X-rays of the first energy level; and
the second sub-imager is configured to receive the X-rays of the second energy level that pass through the site to be treated, and to generate X-ray images of the second energy level of the site to be treated according to the X-rays of the second energy level.

7. The radiotherapy apparatus according to claim 1, wherein the rotating gantry includes any one of a ring gantry and a C-shaped gantry.

8. The radiotherapy apparatus according to claim 1, wherein the X-rays of at least two energy levels include at least X-rays of a kilovolt level and X-rays of a megavolt level.

9. A radiotherapy apparatus, comprising:
a rotating gantry rotatable about a central axis;
a multi-energy imaging device disposed on the rotating gantry, wherein the multi-energy imaging device includes:
an imaging source; and
an imager; and
at least one processor configured to control the imaging source to obtain at least one target X-ray image in conjunction with the imager, wherein the at least one target X-ray image is at least one of X-ray images of at least one energy level of a site to be treated of a patient; the at least one target X-ray image is registered with at least one pre-stored reference image; and a positional deviation of the site to be treated is obtained according to a result of registering the at least one target X-ray image with the at least one reference image.

10. The radiotherapy apparatus according to claim 9, wherein the imaging source is configured to generate X-rays of at least two energy levels and emit X-rays of at least one energy level in the X-rays of at least two energy levels, so that the X-rays of at least one energy level pass through the site to be treated; and the X-ray of at least one energy level is configured to meet imaging requirements of the site to be treated; and
the imager is configured to receive the X-rays of at least one energy level that pass through the site to be treated, and to generate X-ray images of at least one energy level of the site to be treated according to the X-rays of at least one energy level.

11. The radiotherapy apparatus according to claim 10, wherein the X-rays of at least two energy levels include at least X-rays of a first energy level and X-rays of a second energy level;
the radiotherapy apparatus further comprises an imaging source control device, the imaging source includes at least two sub-imaging sources, and the at least two sub-imaging sources include at least a first sub-imaging source and a second sub-imaging source; the first sub-imaging source is configured to generate the X-rays of the first energy level, and the second sub-imaging source is configured to generate the X-rays of the second energy level; and the imaging source control device is configured to control switching of sub-imaging sources that emit X-rays, which are to pass through the site to be treated; or,
the imager includes at least a first sub-imager and a second sub-imager; the first sub-imager is configured to receive the X-rays of the first energy level that pass through the site to be treated, and to generate X-ray images of the first energy level of the site to be treated according to the X-rays of the first energy level; and the second sub-imager is configured to receive the X-rays of the second energy level that pass through the site to be treated, and to generate X-ray images of the second energy level of the site to be treated according to the X-rays of the second energy level.

12. The radiotherapy apparatus according to claim 10, wherein the X-rays of at least two energy levels include at least X-rays of a first energy level and X-rays of a second energy level;
the imaging source includes at least two sub-imaging sources, and the at least two sub-imaging sources include at least a first sub-imaging source and a second sub-imaging source; the imager includes at least two sub-imagers, and the at least two sub-imagers include at least a first sub-imager and a second sub-imager;
the first sub-imaging source and the first sub-imager are arranged opposite to each other on the rotating gantry, and the second sub-imaging source and the second sub-imager are arranged opposite to each other on the rotating gantry;
a line connecting a position of the first sub-imaging source on the rotating gantry and a position of the first sub-imager on the rotating gantry intersects a line connecting a position of the second sub-imaging source on the rotating gantry and a position of the second sub-imager on the rotating gantry;
the first sub-imaging source is configured to generate the X-rays of the first energy level and emit the X-rays of the first energy level, which are to pass through the site to be treated and then reach the first sub-imager, so that the first sub-imager generates X-ray images of the first energy level of the site to be treated according to the X-rays of the first energy level; and
the second sub-imaging source is configured to generate the X-rays of the second energy level and emit the X-rays of the second energy level, which are to pass through the site to be treated and then reach the second sub-imager, so that the second sub-imager generates X-ray images of the second energy level of the site to be treated according to the X-rays of the second energy level.

13. The radiotherapy apparatus according to claim 9, wherein the imaging source is configured to generate X-rays and emit the X-rays, so that the X-rays pass through the site to be treated of the patient; and
the imager is a multi-layer flat plate detector, and is configured to receive the X-rays that pass through the site to be treated and to generate X-ray images of at least two energy levels of the site to be treated.

14. The radiotherapy apparatus according to claim 9, wherein the multi-layer flat plate detector is a dual-layer flat plate detector.

15. A control method of a radiotherapy apparatus, comprising:
controlling an imaging source to obtain at least one target X-ray image in conjunction with an imager, the at least one target X-ray image being at least one of X-ray images of at least one energy level of a site to be treated of a patient;
registering the at least one target X-ray image with at least one pre-stored reference image; and
obtaining a positional deviation of the site to be treated according to a result of registering the at least one target X-ray image with the at least one reference image.

16. The control method of the radiotherapy apparatus according to claim 15, wherein controlling the imaging source to obtain the at least one target X-ray image in conjunction with the imager includes:
controlling the imaging source to emit X-rays of at least two energy levels, which are to pass through the site to be treated, so that the imager generates X-ray images of at least two energy levels according to the X-rays of at least two energy levels that pass through the site to be treated; and
selecting the at least one target X-ray image from the X-ray images of at least two energy levels according to pre-stored information of the site to be treated;
or,
selecting, according to pre-stored information of the site to be treated, X-rays of a target energy level from X-rays of at least two energy levels generated by the imaging source; and
controlling the imaging source to emit the X-rays of the target energy level, so that the imager generates the at least one target X-ray image according to the X-rays of the target energy level that pass through the site to be treated;
or,
controlling the imaging source to emit X-rays, which are to pass through the site to be treated, so that the imager generates X-ray images of at least two energy levels according to the X-rays that pass through the site to be treated; and
selecting the at least one target X-ray image from the X-ray images of at least two energy levels according to pre-stored information of the site to be treated.

17. The control method of the radiotherapy apparatus according to claim 16, further comprising:
controlling a rotating gantry to rotate to at least two different imaging angles, so that there are at least two X-ray images corresponding to different imaging angles in X-ray images generated by the imager according to X-rays of each energy level when the imaging source is controlled to emit preset X-rays, which are to pass through the site to be treated of the patient, wherein
the preset X-rays are the X-rays of at least two energy levels or the X-rays of the target energy level.

18. The control method of the radiotherapy apparatus according to claim 15, wherein before the radiotherapy apparatus is used in treating the patient, the positional deviation includes a setup error; and after the positional deviation of the site to be treated is obtained according to the at least one target X-ray image and the at least one pre-stored reference image, the control method further comprises: controlling the radiotherapy apparatus to adjust a position of the patient according to the positional deviation;
when the radiotherapy apparatus is being used in treating the patient, the positional deviation includes a positional deviation of a tumor in the site to be treated; and after the positional deviation of the site to be treated is obtained according to the at least one target X-ray image and the at least one pre-stored reference image, the control method further comprises: performing deviation correction according to the at least one target X-ray image in a case where it is determined that the positional deviation of the tumor is not within a preset deviation range.

19. The control method of the radiotherapy apparatus according to claim 15, wherein registering the at least one target X-ray image with the at least one pre-stored reference image includes:
processing the at least one target X-ray image; and
registering at least one processed target X-ray image with the at least one reference image.

20. A control apparatus of a radiotherapy apparatus, the control apparatus comprising: a memory, at least one processor, at least one bus, and a communication interface; the memory is configured to store computer-executable instructions, and the at least one processor and the memory are connected through the at least one bus; the at least one processor is configured to execute the computer-executable instructions stored in the memory to cause the control apparatus of the radiotherapy apparatus to perform the control method of the radiotherapy apparatus according to claim 9.
